(19) United States
(12) Patent Application Publication
(10) Pub. No.: US 2026/0007327 A1
(43) Pub. Date: Jan. 8, 2026

(54) ADAPTER, MASK, AND SYSTEM FOR DETECTING CONCENTRATION OF CARBON DIOXIDE

(71) Applicant: NIHON KOHDEN CORPORATION, Tokyo (JP)

(72) Inventors: Naoki KOBAYASHI, Saitama (JP); Kota SAEKI, Saitama (JP); Yuya BABA, Saitama (JP); Fumihiko TAKATORI, Saitama (JP); Shinji YAMAMORI, Saitama (JP)

(73) Assignee: NIHON KOHDEN CORPORATION, Tokyo (JP)

(21) Appl. No.: 19/251,071

(22) Filed: Jun. 26, 2025

(30) Foreign Application Priority Data
Jul. 5, 2024 (JP) 2024-108661

(51) Int. Cl. A61B 5/08; A61B 5/00; A61M 16/06; A61M 16/08

(52) U.S. Cl. CPC A61B 5/082; A61B 5/0082; A61M 16/0611; A61M 16/085; A61M 2230/43

(57) ABSTRACT

An adapter is configured to be coupled with a mask that is adapted to be attached on a face of a subject to supply air or oxygen gas to an airway of the subject. A base defines a ventilation path adapted to be communicated with an exhaust hole formed in the mask. A retainer is configured to removably retain a sensor including a light detecting element configured to output a signal corresponding to concentration of carbon dioxide as detected optically. The ventilation path is located on a path of light that is to be incident on the light detecting element.

### CROSS-REFERENCE TO RELATED APPLICATION

The present application is based on Japanese Patent Application No. 2024-108661 filed on Jul. 5, 2024, the entire contents of which are incorporated herein by reference.

### BACKGROUND

The presently disclosed subject relates to an adapter configured to be coupled with a mask that is adapted to be coupled with a face of a subject to supply air or oxygen gas to an airway of the subject. The presently disclosed subject matter also relates to a mask coupled with the adapter, as well as a system for detecting a concentration of carbon dioxide contained in respiratory gas of a subject using the mask.

International Publication No. WO 2021/257836 A1 discloses a device for detecting a respiratory state of a subject. The device is provided with an indicator that changes color in accordance with concentration of carbon dioxide. The device is coupled with an exhaust hole formed in a mask for supplying air or oxygen gas to an airway of the subject. The color of the indicator reflects the concentration of carbon dioxide contained in respiratory gas of the subject that passes through the exhaust hole. The color of the indicator is evaluated visually.

### SUMMARY

It is required to quantitatively evaluate the concentration of carbon dioxide contained in the respiratory gas of a subject while suppressing degradation of the versatility and popularity of the mask.

An illustrative aspect of the presently disclosed subject matter may provide an adapter configured to be coupled with a mask that is adapted to be attached on a face of a subject to supply air or oxygen gas to an airway of the subject, the adapter comprising:
a base defining a ventilation path adapted to be communicated with an exhaust hole formed in the mask; and
a retainer configured to removably retain a sensor including a light detecting element configured to output a signal corresponding to concentration of carbon dioxide as detected optically,
wherein the ventilation path is located on a path of light that is to be incident on the light detecting element.

A capnograph is known as a technique for detecting the concentration of carbon dioxide contained in the respiratory gas. In the capnograph, it is used a sensor including a light emitting element that emits infrared light, and a light detecting element that outputs a signal corresponding to intensity of light as detected. Such a sensor is coupled with a mask having a ventilation path formed in a path of the infrared light that is to be incident on the light detecting element, and configured to allow the expiration gas of the subject to flow. The infrared light is absorbed by the carbon dioxide contained in the expiration gas of the subject that flows through the ventilation path. Accordingly, the signal outputted from the light detecting element reflects the concentration of carbon dioxide. The mask is generally provided as a dedicated product provided with a structure for retaining such a sensor in a portion where the ventilation path is formed.

According to the configuration of the above illustrative aspect, the ventilation path is formed in the adapter provided with the retainer that removably retains the sensor. The adapter is coupled with the mask such that the exhaust holes formed in the mask are communicate with the ventilation path. As a result, the detection system for quantitatively evaluating the concentration of carbon dioxide contained in the respiratory gas of the subject can be configured. On the other hand, a typical mask used for supplying air or oxygen gas to the airway of a subject generally includes exhaust holes for exhausting the expiration gas of the subject as well as the air or oxygen gas that is excessively supplied. Accordingly, the mask with which the adapter is coupled may be a non-dedicated product. Since it is not necessary to provide an additional structure on the side of the mask in order to configure the detection system, it is possible to suppress degradation of the versatility and the popularity of the mask.

An illustrative aspect of the presently disclosed subject matter may provide a mask adapted to be attached on a face of a subject to supply air or oxygen gas to an airway of the subject, the mask comprising:
a body section formed with at least one exhaust hole; and
an adapter disposed on the body section so as to cover the exhaust hole,
wherein the adapter includes:
   a base defining a ventilation path adapted to be communicated with an exhaust hole formed in the mask; and
   a retainer configured to removably retain a sensor including a light detecting element configured to output a signal corresponding to concentration of carbon dioxide as detected optically,
wherein the ventilation path is located on a path of light that is to be incident on the light detecting element.

An illustrative aspect of the presently disclosed subject matter may provide a system for detecting concentration of carbon dioxide, comprising:
a mask adapted to be attached on a face of a subject to supply air or oxygen gas to an airway of the subject;
an adapter disposed on the mask so as to cover at least one exhaust hole formed in the mask; and
a sensor including a light detecting element configured to output a signal corresponding to concentration of carbon dioxide as detected optically,
wherein the adapter includes:
   a base defining a ventilation path adapted to be communicated with an exhaust hole formed in the mask; and
   a retainer configured to removably retain the sensor,
wherein the ventilation path is located on a path of light that is to be incident on the light detecting element.

### DESCRIPTION OF THE DRAWINGS

FIG. 1 illustrates an appearance of a mask according to an exemplary embodiment.

FIG. 2 illustrates an appearance of a system for detecting concentration of carbon dioxide according to an exemplary embodiment.

FIG. 3 illustrates an appearance of components of the system of FIG. 2.

FIG. 4 illustrates an appearance of an adapter as viewed from a direction of an arrow IV of FIG. 3.

FIG. 5 illustrates an appearance of the adapter as viewed from a direction of an arrow V of FIG. 4.

FIG. 6 illustrates an appearance of the adapter as viewed from a direction of an arrow VI of FIG. 5.

FIG. 7 illustrates a state where a holder of FIG. 3 is supported by a support.

FIG. 8 illustrates a cross section of the adapter along a line VIII-VIII of FIG. 4 as viewed from an arrowed direction.

FIG. 9 illustrates a cross section of the adapter along a line IX-IX of FIG. 4 as viewed from an arrowed direction.

FIG. 10 illustrates an appearance of the adapter as viewed from a direction of an arrow X of FIG. 9.

FIG. 11 illustrates another exemplary appearance of the adapter.

### DESCRIPTION OF EMBODIMENTS

Examples of embodiments will be described below in detail with reference to the accompanying drawings. In each of the drawings, the scale is appropriately changed in order to make each element as illustrated have a recognizable size.

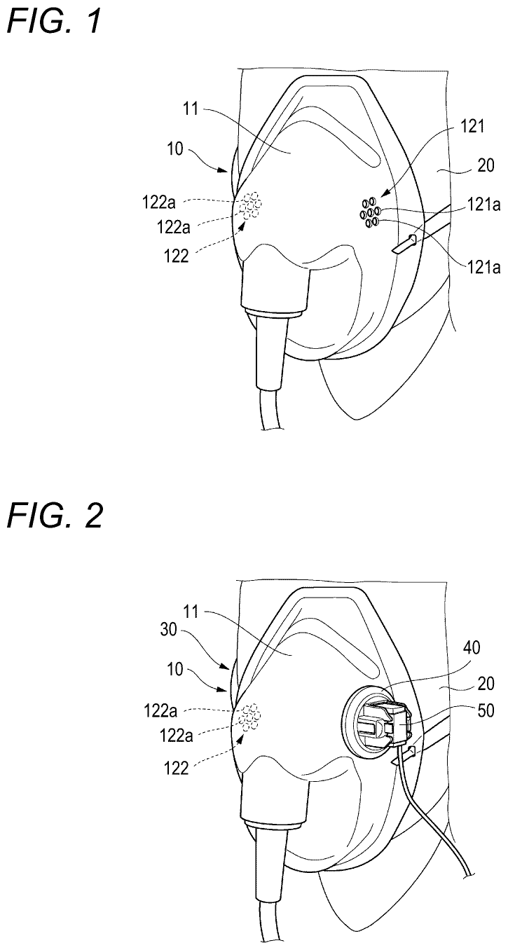

FIG. 1 illustrates an appearance of a mask 10 according to an exemplary embodiment. The mask 10 is adapted to be attached to a face 20 of a subject to supply air or oxygen gas to an airway of the subject. The mask 10 includes a body section 11 defining a space adapted to cover a nose and a mouth of the subject.

The body section 11 includes a first exhausting portion 121. The first exhausting portion 121 is located on the right of the center of the body section 11 relative to a left-right direction that is corresponding to a left-right direction of the face 20. The first exhausting portion 121 includes multiple exhaust holes 121a that are formed so as to penetrate the body section 11. In other words, the space defined by the body section 11 communicates with ambient air through the multiple exhaust holes 121a.

FIG. 2 illustrates an appearance of a system 30 for detecting concentration of carbon dioxide (hereinafter, referred to as “detection system 30”) according to an exemplary embodiment. The detection system 30 is configured to detect a concentration of carbon dioxide contained in respiratory gas of the subject. In addition to the mask 10 described above, the detection system 30 includes an adapter 40 and a sensor 50.

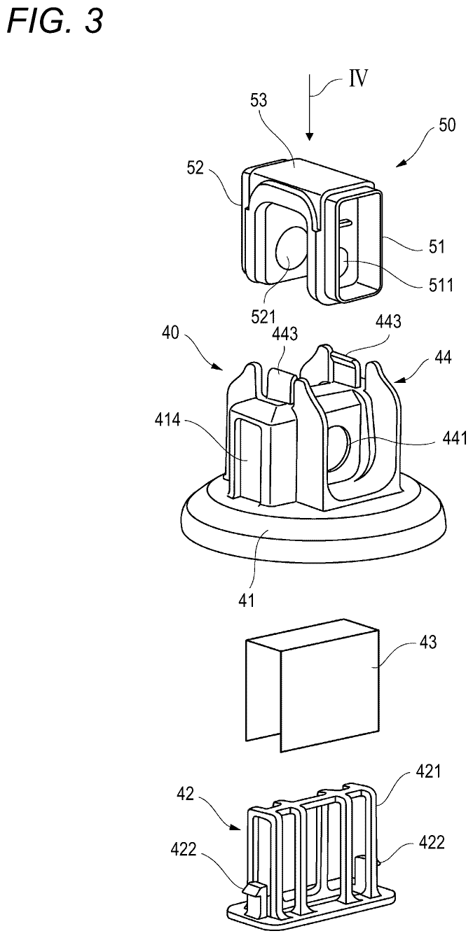

The adaptor 40 is coupled with the body section 11 of the mask 10 so as to cover the first exhausting portion 121. In this example, all the exhaust holes 121a are covered by the adaptor 40. The sensor 50 is held by the adapter 40. The sensor 50 is configured to optically detect a concentration of carbon dioxide. As illustrated in FIG. 3, the sensor 50 is detachable from the adapter 40.

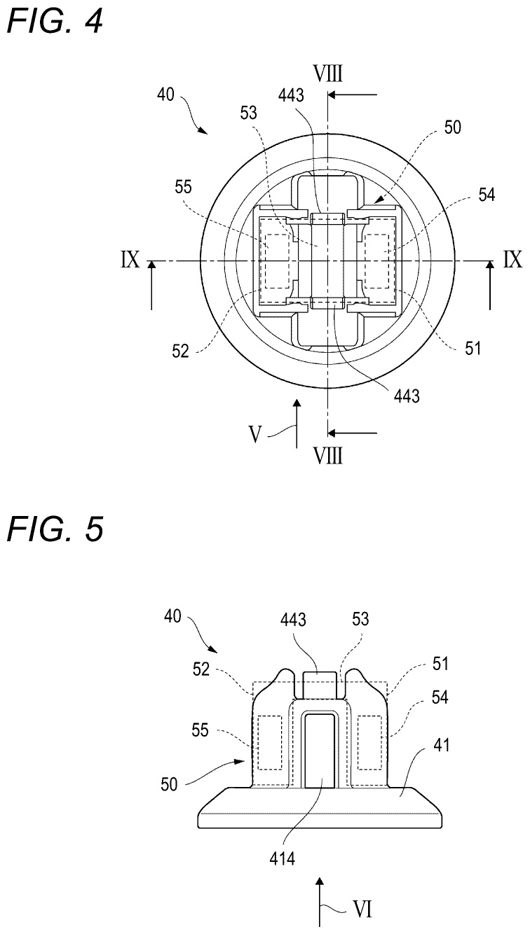

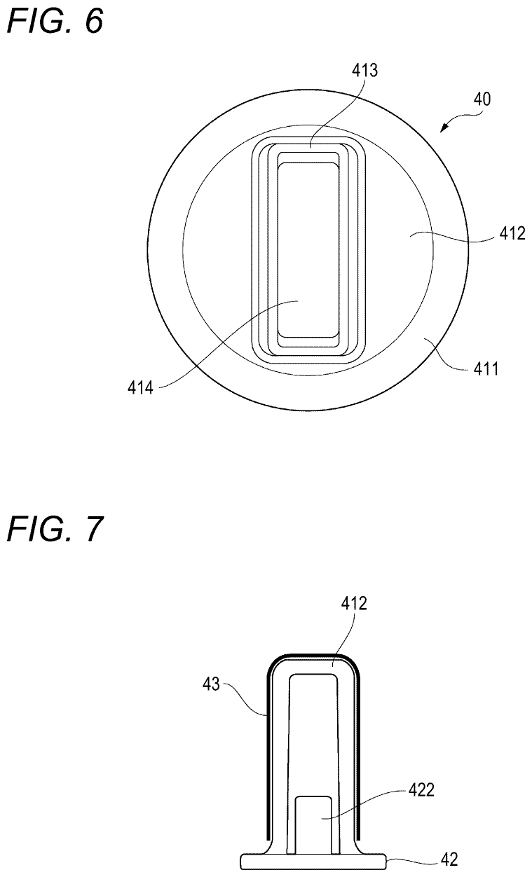

FIG. 4 illustrates an appearance of the adapter 40 as viewed from the direction of the arrow IV in FIG. 3. FIG. 5 illustrates an appearance of the adapter 40 as viewed from the direction of the arrow V in FIG. 4. FIG. 6 illustrates an appearance of the adapter 40 as viewed from the direction of arrow VI in FIG. 5.

The adapter 40 includes a base 41. As illustrated in FIG. 6, the base 41 has a bottom face 411. As the adapter 40 is coupled with the mask 10, the bottom face 411 faces the first exhausting portion 121. The bottom face 411 has a recess 412. The recess 412 has a shape and a size that can cover the multiple exhaust holes 121a. In other words, as the adaptor 40 is coupled with the mask 10, the recess 412 defines a space communicating with each of the multiple exhaust holes 121a. An opening 413 is formed in the bottom of the recess 412. The opening 413 is opened in a space defined by the recess 412.

As illustrated in FIG. 5, the base 41 defines a laterally open ventilation path 414. The ventilation path 414 communicates with the opening 413. In other words, as the adaptor 40 is coupled with the mask 10, the ventilation path 414 communicates with the multiple exhaust holes 121a.

As illustrated in FIG. 3, the adapter 40 includes a support 42 and a holder 43. As illustrated in FIG. 7, the support 42 has a protrusion 421 for supporting the holder 43. The holder 43 is a sheet-shaped member that holds liquid containing dye. The holding of the liquid is realized by immersing the holder 43 in the liquid. An exemplary composition of the liquid is as follows.
100 mL of 0. 01M aqueous sodium carbonate solution
50 mL of glycerin
60 mg of metacresol purple sodium salt
80 mL of purified water

As the carbon dioxide contacts the liquid and dissolves in the liquid phase, the pH value of the liquid is decreased. The dye according to this example has a property of changing color in accordance with the pH value. Specifically, the dye has a purple color in a high-pH region, and a yellow color in a low-pH region.

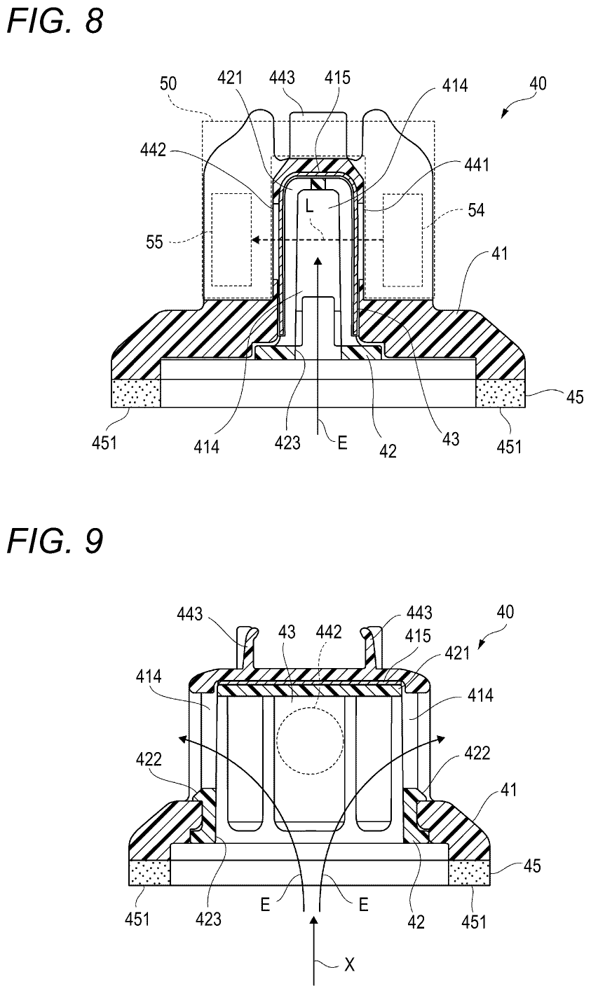

FIG. 8 illustrates a cross-section of the adapter 40 along a line VIII-VIII in FIG. 4 as viewed from an arrowed direction. FIG. 8 illustrates a cross-section of the adapter 40 along a line IX-IX in FIG. 4 as viewed from an arrowed direction. The above-described ventilation path 414 is defined by a recess 415 formed in the interior of the base 41. The protrusion 421 of the support 42 that supports the holder 43 is inserted into the recess 415 from the opening 413 formed in the base 41.

As illustrated in FIG. 3, the support 42 is provided with an engagement pawl 422. As the protrusion 421 is inserted to a prescribed depth, the engagement pawl 422 engages with the base 41 as illustrated in FIG. 9. Accordingly, the support 42 is coupled to the base 41. As illustrated in FIGS. 8 and 9, in this state, the holder 43 is clamped between the recess 415 and the protrusion 421.

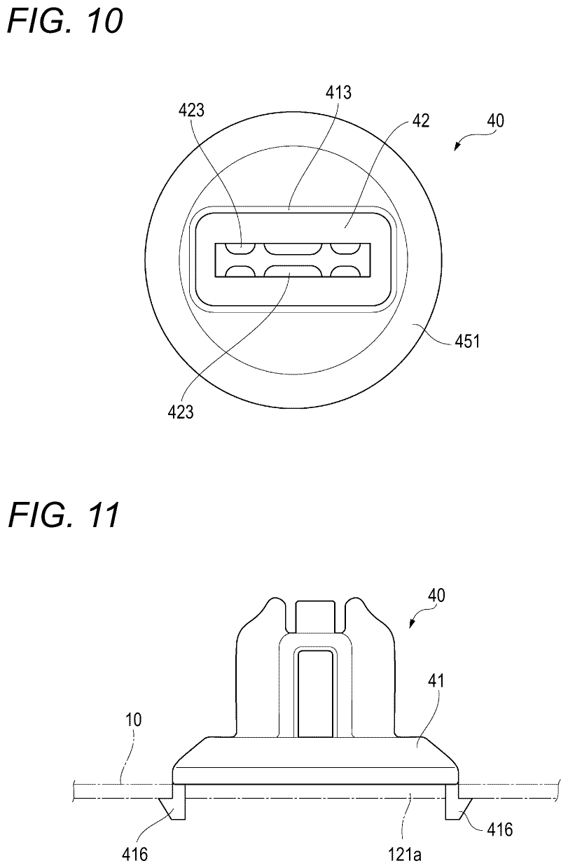

FIG. 10 illustrates an appearance of the adapter 40 as viewed from the direction of the arrow X in FIG. 9. The support 42 has multiple openings 423. As the support 42 is coupled to the base 41, each of the multiple openings 423 communicates with the ventilation path 414.

As illustrated in FIGS. 3 to 5, the sensor 50 has a first portion 51, a second portion 52, and a third portion 53. The first portion 51 defines a space for accommodating a light emitting element 54. The second portion 52 defines a space for accommodating a light detecting element 55. The third portion 53 supports the first portion 51 and the second portion 52 so as to face each other with a gap therebetween.

The first portion 51 has a window 511. The second portion 52 has a window 521. The window 511 and the window 521 are disposed so as to face each other with the above-described gap therebetween. The window 511 is configured to allow passage of the light emitted from the light emitting element 54. The window 521 is configured to allow passage of the light that is to be incident on the detection face of the light detecting element 55.

The adaptor 40 includes a retainer 44. The retainer 44 is configured to removably retain the sensor 50. As illustrated in FIGS. 3 and 8, the retainer 44 includes a first window 441, a second window 442, and an engagement pawl 443. The first window 441 is configured to allow passage of the light emitted from the light emitting element 54. The second window 442 is configured to allow passage of the light that is to be incident on the light detection face of the light detecting element 55.

As the sensor 50 is coupled with the adapter 40, the engagement pawl 443 engages with the third portion 53. Accordingly, detachment of the sensor 50 is from the adapter 40 is prevented. In this state, the window 511 of the first portion 51 is disposed so as to face the first window 441. Similarly, the window 521 of the second portion 52 is disposed so as to face the second window 442. As a result, the ventilation path 414 is located on the path of the light L that is emitted from the light emitting element 54 and to be incident on the light detecting element 55. The holder 43 supported by the support 42 and disposed in the ventilation path 414 is also located on the path of the light L.

The wavelength of the light beam Lis selected such that the discoloration of the above-described dye can be detected. In this example, the wavelength is appropriately selected in the visible light range. The central wavelength may be multiple.

Expiration gas of the subject is exhausted through the exhaust holes 121a formed in the body section 11 of the mask 10. As illustrated in FIGS. 8 and 9, the expiration gas E enters the ventilation path 414 of the adapter 40 through the openings 423 formed in the support 42. Thereafter, the expiration gas E is exhausted to a lateral side of the base 41 while contacting with the holder 43 supported by the support 42.

In accordance with the contact between the expiration gas E and the holder 43, the color of the dye contained in the liquid held by the holder 43 is changed in accordance with the pH value corresponding to the concentration of the carbon dioxide contained in the expiration gas E. Since the concentration of carbon dioxide contained in the expiration gas E is relatively high, the color of the holder 43 turns yellow in this example.

Since the light L emitted from the light emitting element 54 passes through the holder 43, the intensity of the wavelength component detected by the light detecting element 55 is changed in accordance with the color of the holder 43. Since the light detecting element 55 is configured to output a signal corresponding to the intensity, the signal also changes in accordance with the color of the holder 43. In other words, the light detecting element 55 outputs a signal corresponding to the concentration of carbon dioxide that is optically detected.

The signal is inputted to an adequate signal processing device, whereby the signal is subjected to quantitative evaluation of the concentration of carbon dioxide contained in the respiratory gas of the subject. As an example, based on the fact that a decrease in the concentration of carbon dioxide contained in the respiratory gas is detected, an evaluation that the subject is in a condition of respiratory depression may be performed.

A capnograph is known as a technique for detecting the concentration of carbon dioxide contained in the respiratory gas. In the capnograph, it is used a sensor including a light emitting element that emits infrared light, and a light detecting element that outputs a signal corresponding to intensity of light as detected. Such a sensor is coupled with a mask having a ventilation path formed in a path of the infrared light that is to be incident on the light detecting element, and configured to allow the expiration gas of the subject to flow. The infrared light is absorbed by the carbon dioxide contained in the expiration gas of the subject that flows through the ventilation path. Accordingly, the signal outputted from the light detecting element reflects the concentration of carbon dioxide. The mask is generally provided as a dedicated product provided with a structure for retaining such a sensor in a portion where the ventilation path is formed.

In the present exemplary embodiment, the ventilation path 414 is formed in the adapter 40 provided with the retainer 44 that removably retains the sensor 50. The adapter 40 is coupled with the mask 10 such that the exhaust holes 121a formed in the mask 10 are communicate with the ventilation path 414. As a result, the detection system 30 for quantitatively evaluating the concentration of carbon dioxide contained in the respiratory gas of the subject can be configured. On the other hand, a typical mask used for supplying air or oxygen gas to the airway of a subject generally includes exhaust holes for exhausting the expiration gas of the subject as well as the air or oxygen gas that is excessively supplied. Accordingly, the mask 10 with which the adapter 40 is coupled may be a non-dedicated product. Since it is not necessary to provide an additional structure on the side of the mask 10 in order to configure the detection system 30, it is possible to suppress degradation of the versatility and the popularity of the mask 10.

In addition, in the present exemplary embodiment, the holder 43 holding the liquid containing the dye whose color changes in accordance with the pH value corresponding to the concentration of carbon dioxide is disposed in the ventilation path 414 of the adapter 40. According to such a configuration, the concentration of carbon dioxide can be optically detected with a sensor that uses a visible wavelength range. Since such a sensor can be obtained at a lower cost than a sensor using infrared light, it is possible to further suppress degradation of the versatility and popularity.

In addition, in the present exemplary embodiment, the support 42 has the protrusion 421 that supports the holder 43, and the ventilation path 414 is defined by the recess 415 formed in the base 41. The support 42 is coupled with the base 41 such that the protrusion 421 is inserted into the recess 415, whereby the holder 43 is supported between the protrusion 421 and the recess 415.

According to such a configuration, the holder 43 is placed on the support 42 so as to extend along the protrusion 421, and the protrusion 421 is inserted into the recess 415. As a result, the holder 43 can be easily disposed at a prescribed position in the ventilation path 414. In addition, since the holder 43 holds liquid containing glycerin, fixation with adhesion is difficult. However, according to the configuration of this example, the holder 43 can be easily fixed to the prescribed position, and deregistration can be suppressed. Accordingly, it is possible to suppress degradation of the detection accuracy for the concentration of carbon dioxide while improving the workability for configuring the detection system 30.

In this example, as illustrated in FIG. 7, the holder 43 is disposed along the upper face and both side faces of the protrusion 421. However, as long as the holder 43 can be supported between the protrusion 421 and the recess 415 when the support 42 is attached to the base 41, the holder 43 may be disposed so as to extend along at least one of the both side faces of the protrusion 421.

It should be noted that there is no intention to exclude a case where a sensor for capnograph using infrared light is retained by the retainer 44 of the adapter 40 as the sensor 50. In this case, the light emitting element and the light detecting element are disposed so that the infrared light passes through the ventilation path 414. The support 42 and the holder 43 are omitted.

As illustrated in FIGS. 8 and 9, the adapter 40 may include a cushion part 45. The cushion part 45 is formed of a material having higher flexibility than the base 41 and the retainer 44. The cushion part 45 has a contact face 451 that can contact an outer face of the mask 10.

The outer face of the mask 10 is generally curved. In addition, in a case where coupling with the mask 10 as the non-dedicated product is supposed, the shape of the outer face with which the adapter 40 is coupled may not be always the same. According to the above configuration, since the flexibility of the cushion part 45 compensates the difference in shape between the flat bottom face 411 of the base 41 and the non-flat outer face of the mask 10, it is possible to suppress the formation of a gap between the adapter 40 and the mask 10. Accordingly, it is possible to suppress occurrence of a case where the expiration gas E of the subject leaks from such a gap. As a result, it is possible to suppress degradation of the detection accuracy for the concentration of carbon dioxide contained in the respiratory gas of the subject.

In this example, the contact face 451 has adhesiveness. Accordingly, as the contact face 451 is just brought into contact the outer face of the body section 11 of the mask 10, the coupling of the adapter 40 with the mask 10 is finished. As a result, the workability for configuring the detection system 30 can be improved.

As illustrated in FIG. 11, the base 41 of the adapter 40 may include an engagement member 416 in accordance with the size and shape of the exhaust hole 121a formed in the body section 11 of the mask 10. The engagement member 416 is configured to mechanically engage with the exhaust hole 121a.

In this case, as the engagement member 416 is just engaged with the exhaust hole 121a of the mask 10, the coupling of the adapter 40 with the mask 10 is finished. Accordingly, the workability for configuring the detection system 30 can be improved.

It should be noted that the cushion part 45 may be provided at a position where the mechanical coupling of the engagement member 416 to the exhaust hole 121a is not interfered. In this case, the contact face 451 of the cushion part 45 may or may not have the adhesiveness.

In the present exemplary embodiment, the ventilation path 414 of the adapter 40 is formed such that a channel cross-section thereof is larger than a channel cross-section of the first exhausting portion 121 formed in the mask 10. The “channel cross-section of the first exhausting portion 121” is defined as a sum of the areas of the multiple exhaust holes 121a. In the present exemplary embodiment, the channel cross-section of the ventilation path 414 at a place where the multiple openings 423 are provided in the support 42 is minimum. At that place, the “channel cross-section of the ventilation path 414” is defined as a sum of the areas of the multiple openings 423. Even at that place, the channel cross-section of the ventilation path 414 is larger than the channel cross-section of the first exhausting portion 121. In other words, a flow resistance of the ventilation path 414 of the adapter 40 is less than a flow resistance of the first exhausting portion 121 of the mask 10.

According to such a configuration, it is possible to prevent the coupling of the adapter 40 with the mask 10 from degrading the inherent ventilating function of the mask 10. Accordingly, it is possible to further suppress the degradation in the versatility and popularity of the mask 10.

It should be noted that, as long as the above-described relationship related to the channel cross-section is satisfied, it is not necessary to cover all of the multiple exhaust holes 121a with the adaptor 40.

As illustrated in FIG. 1, the mask 10 includes a second exhausting portion 122. The second exhausting portion 122 is located on the left of the center of the body section 11 in the left-right direction corresponding to the left-right direction of the face 20. The second exhausting portion 122 includes multiple exhaust holes 122a that are formed so as to penetrate the body section 11. In other words, the space defined by the body section 11 communicates with the ambient air through the multiple exhaust holes 122a.

As illustrated in FIG. 2, the second exhausting portion 122 is not covered with the adapter 40. In other words, the adapter 40 is coupled with the mask 10 such that exhaust holes 122a are exposed.

According to such a configuration as well, it is possible to prevent the coupling of the adapter 40 with the mask 10 from degrading the inherent ventilating function of the mask 10. Accordingly, it is possible to further suppress the degradation in the versatility and popularity of the mask 10.

Each of the configurations exemplified above is merely illustrative for facilitating understanding of the presently disclosed subject matter. Each exemplary configuration may be appropriately modified or combined with another exemplary configuration within the scope of the presently disclosed subject matter.

In the above exemplary embodiment, the adapter 40 coupled with the mask 10 is provided independently from the mask 10. However, a mask 10 with which the adapter 40 has been coupled may be provided.

1. An adapter configured to be coupled with a mask that is adapted to be attached on a face of a subject to supply air or oxygen gas to an airway of the subject, the adapter comprising:
a base defining a ventilation path adapted to be communicated with an exhaust hole formed in the mask; and
a retainer configured to removably retain a sensor including a light detecting element configured to output a signal corresponding to concentration of carbon dioxide as detected optically,
wherein the ventilation path is located on a path of light that is to be incident on the light detecting element.

2. The adapter according to claim 1, further comprising:
a holder holding liquid containing dye a color of which changes in accordance with a pH value corresponding to the concentration of carbon dioxide,
wherein the holder is located on the path of light.

3. The adapter according to claim 2, further comprising:
a support provided with a protrusion configured to support the holder,
wherein the ventilation path is defined by a recess formed in the base; and
wherein as the support is attached to the base such that the protrusion is inserted into the recess, the holder is clamped between the protrusion and the recess.

4. The adapter according to claim 1, further comprising:
a cushion part adapted to contact an outer face of the mask, and having higher flexibility than the base and the retainer.

5. The adapter according to claim 4,
wherein the cushion part has adhesiveness with respect to the outer face of the mask.

6. The adapter according to claim 1, further comprising:
an engagement member adapted to mechanically engage with the exhaust hole.

7. The adapter according to claim 1,
wherein a channel cross-section of the ventilation path is larger than a channel cross-section of the exhaust hole.

8. A mask adapted to be attached on a face of a subject to supply air or oxygen gas to an airway of the subject, the mask comprising:
a body section formed with at least one exhaust hole; and
an adapter disposed on the body section so as to cover the exhaust hole,
wherein the adapter includes:
a base defining a ventilation path adapted to be communicated with an exhaust hole formed in the mask; and
a retainer configured to removably retain a sensor including a light detecting element configured to output a signal corresponding to concentration of carbon dioxide as detected optically,
wherein the ventilation path is located on a path of light that is to be incident on the light detecting element.

9. The mask according to claim 8,
wherein the body section is formed with multiple exhaust holes; and
wherein the adapter is attached such that at least one of the exhaust holes is exposed.

10. A system for detecting concentration of carbon dioxide, comprising:
a mask adapted to be attached on a face of a subject to supply air or oxygen gas to an airway of the subject;
an adapter disposed on the mask so as to cover at least one exhaust hole formed in the mask; and
a sensor including a light detecting element configured to output a signal corresponding to concentration of carbon dioxide as detected optically,
wherein the adapter includes:
a base defining a ventilation path adapted to be communicated with an exhaust hole formed in the mask; and
a retainer configured to removably retain the sensor,
wherein the ventilation path is located on a path of light that is to be incident on the light detecting element.

11. The system according to claim 10,
wherein the signal corresponds to a color of dye that changes in accordance with a pH value corresponding to the concentration of carbon dioxide.

12. The system according to claim 10,
wherein the signal corresponds to intensity of infrared light incident on the light detecting element.
